(19) United States
(12) Patent Application Publication
(10) Pub. No.: US 2023/0227392 A1
(43) Pub. Date: Jul. 20, 2023

(54) A APPLICATION OF CYATHANE DITERPENOIDS IN PREPARATION OF DRUGS FOR TREATING NEUROINFLAMMATION

(71) Applicant: Northwest A&F University, Xianyang (CN)

(72) Inventors: JinMing Gao, Xianyang (CN); Chenyu Cao, Xianyang (CN)

(21) Appl. No.: 18/089,567

(22) Filed: Dec. 28, 2022

(30) Foreign Application Priority Data
Jan. 17, 2022 (CN) 2022100504065

(51) Int. Cl. C07C 47/46; C07C 69/78; C07C 69/16; A61P 29/00; A61P 25/28

(52) U.S. Cl. CPC C07C 47/46; C07C 69/78; C07C 69/16; A61P 29/00; A61P 25/28

(57) ABSTRACT

The present invention discloses the application of cyathane diterpenoids in preparation of drugs for treating neuroinflammation. Seven cyathane diterpenoids 1-7 can significantly prevent LPS-induced BV-2 microglia from producing NO. The most active compound as 6 can reverse M1/M2 polarization of the microglia. 6 inhibits the pro-inflammatory proteins including iNOS and COX-2 through MAPK/NF-κB signaling pathways and promote the anti-inflammatory factors including IL-10 and ARG-1. An application prospect of the anti-inflammatory cyathane diterpenoids in the treatment of the neurodegenerative diseases is achieved.

### TECHNICAL FIELD

The present invention belongs to the field of drugs, and relates to the application of cyathane diterpenoids in preparation of drugs for treating neuroinflammation.

### BACKGROUND

With the development of aging society, neurodegenerative diseases (ND) have become global public health and social problems. ND mainly include Alzheimer's disease (AD), amyotrophic lateral sclerosis (ALS), Huntington's disease (HD) and Parkinson's disease (PD). Among them, AD have the highest incidence rate. It has been listed as one of the five major diseases that seriously threaten human health in the 21st century by WHO. Every three seconds, one person is diagnosed with AD. The International Alzheimer's Association estimated that more than 152 million person worldwide will have AD by 2050 and the global AD-related cost will rise to 1 trillion dollars by 2030. In China, there have been more than 10 million patients with AD, ranking first in the world. Therefore, the development of safe and effective drugs for the prevention and treatment of ND have become one of the hotspots in the field of drugs in China and even in the world^{[1]}.

AD is a neurodegenerative disease with complicated pathogenesis. The main pathological features are β-amyloid (Aβ) plaque deposition, intracellular neurofibrillary tangles (NFTs), oxidative stress, neuroinflammation, neurotransmitter deficiency, etc.^{[2]}. More and more evidence indicated that an interaction between the neuroinflammation and the pathological amyloid makes tau protein spread, which eventually leads to extensive brain injury and cognitive impairment^{[3]}. Therefore, neuroinflammation could be an indispensable upstream mechanism for the development of AD. Correspondingly, the drugs targeted neuroinflammation are expected to be a promising method for treating AD^{[4]}.

The neuroinflammation is characterized as the activation of the microglia cells which play a vital role in the immune response of CNS. The microglia have dual phenotypes and functional plasticity. Classical phenotype (M1) is characterized by the release of nitric oxide (NO), tumor necrosis factor (TNF-α), interleukin-1β (IL-1β), interleukin-6 (IL-6), inducible nitric oxide synthase (iNOS) and cyclooxygenase-2 (COX-2), while the other phenotype (M2) are characterized by the release of anti-inflammatory molecules, such as interleukin-10 (IL-10) and arginase-1 (ARG-1)^{[5]}. In fact, M2 microglia can create a favorable microenvironment for CNS repair through neurogenesis, axon regeneration, angiogenesis and vascular repair of patients with stroke, AD, Parkinson's disease and other ND. On the contrary, the pro-inflammatory cytokines TNF-α and IL-1β can promote peripheral astrocyte neurons to produce more Aβ42 oligomers, to accelerate the pathological process of AD. Therefore, the fine regulation of M1/M2 polarization have been considered as a more effective therapeutic for neuroinflammatory-associated diseases rather than broad inhibition of the microglia^{[6-7]}.
[1] Mitra S, Behbahani H, Eriksdotter M. 2019. Innovative Therapy for Alzheimer's Disease—With Focus on Biodelivery of NGF. Front Neurosci. 13:38-59.
[2] Fish P V, Steadman D, Bayle E D, Whiting P. 2019. New approaches for the treatment of Alzheimer's disease. Bioorg Med Chem Lett. 29(2):125-133.
[3] Ubiquitin Ligase COP1 Suppresses Neuroinflammation by Degrading c/EBPβin Microglia. Cell. 2020; 182(5):1156-1169.
[4] Webers A, Heneka M T, Gleeson P A. 2020. The role of innate immuneresponses and neuroinflammation in amyloid accumulation and progression of Alzheimer's disease. Immunol Cell Biol. 98(1):28-41.
[5] Orihuela R, McPherson C A, Harry G J. 2016. Microglial M1/M2 polarization and metabolic states. Br J Pharmacol. 173(4):649-665.
[6] Shabab T, Khanabdali R, Moghadamtousi S Z, Kadir H A, Mohan G. 2017. Neuroinflammation pathways: a general review. Int J Neurosci. 127(7):624-633.
[7] Song G J, Suk K. 2017. Pharmacological Modulation of Functional Phenotypes of Microglia in Neurodegenerative Diseases. Front Aging Neurosci. 9:139-149.

### SUMMARY

According to the present invention, a typical cyathane diterpenoid Sarcodonin A is isolated from fruiting bodies of Sarcodon scarbrosus (Fr.) Karst, and is then semi-synthesized to obtain six compounds (2-7). All the compounds can significantly inhibit LPS-induced BV-2 microglia from producing NO and compound 6 has the strongest activity. Further research shows that 6 can promote the down-regulation the inflammatory proteins (iNOS and COX-2) and cytokines (TNF-α, IL-1β and IL-6) through MAPK/NF-κB signaling pathways, and exhibits significant anti-neuroinflammatory activity by reversing the M1/M2 microglia polarization.

The chemical structures of sarcodonin A (1) and its derivatives (2-7) are listed as:

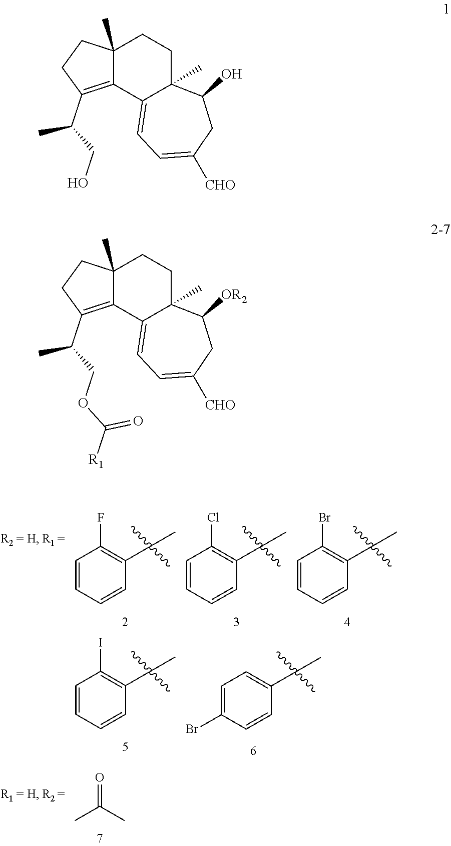

Based on this, the present invention provides the application of the above cyathane diterpenoids or hydrates thereof or pharmaceutically acceptable salts thereof or pharmaceutically acceptable carriers thereof, and compositions containing the cyathane diterpenoids in preparation of drugs for treating neuroinflammatory or neurodegenerative diseases.

The neurodegenerative diseases include but not limited to Alzheimer's disease (AD), amyotrophic lateral sclerosis (ALS), Huntington's disease (HD) and Parkinson's disease (PD), etc.

The compounds according to the present invention can obviously inhibit the LPS-induced BV-2 cells from producing NO at 10 M. The compound 6 can regulate the MAPK/NF-κB signaling pathways and the expression of downstream inflammatory factors, and thus inhibit the neuroinflammation by reversing the M1/M2 microglia polarization.

As neuroprotective small molecules, the cyathane diterpenoids according to the invention have development and application potentials in preparation of drugs for treating neurodegenerative diseases related to neuroinflammation.

### BRIEF DESCRIPTION OF DRAWINGS

The accompanying drawings are configured to provide a further understanding of the present disclosure, and form a part of the description. Together with the following detailed embodiments, the accompanying drawings are configured to explain the present disclosure, but do not constitute a limitation on the present disclosure. In the figures:

FIG. 1 shows effects of compounds 1-7 on vitality of BV-2 cells;

FIG. 2 shows effects of compounds 1-7 on the production of NO of LPS-induced BV-2;

FIG. 3 shows effects of compounds 1 and 6 on mRNA expression of M1 and M2 biomarkers of LPS-induced BV-2 cells;

FIG. 4 shows effects of compounds 1 and 6 on cytokine release of the M1 and M2 markers of the LPS-induced BV-2 cells;

FIG. 5 shows effects of compounds 1 and 6 on protein levels of the M1 and M2 markers of LPS-stimulated BV-2 cells;

FIG. 6 shows effects of compounds 1 and 6 on phosphorylation of ERK1/2, JNK and p38 MAPK and nuclear transfer of NF-κB of the LPS-stimulated BV-2 cells;

FIG. 7 shows the lowest energy conformation of molecular docking simulation 1(a) and 6(b) entering active sites of iNOS, and for an iNOS dimer, A chains are green and B chains are cyan; hydrogen bond interactions are highlighted by yellow dotted lines.

### DETAILED DESCRIPTION OF EMBODIMENTS

Hereinafter, the technical solutions in the embodiments of the present invention will be further explained in detail in combination with the drawings in the embodiments of the present invention. Obviously, the embodiments described below are only part and not all of the embodiments of the present invention, and do not limit the present invention in any form.

Without departing from the spirit and scope of the inventive concept, variations and advantages conceivable by those skilled in the art are included in the present invention, and the appended claims are taken as the scope of protection.

The processes, conditions, reagents, experimental methods and the like for the implementation of the present invention are all general knowledge and common knowledge in the art, except those specifically mentioned below, and the present invention does not particularly limit the content.

The compound cyathane diterpenoid sarcodonin A (1) according to the present invention is isolated from fruiting bodies of Sarcodon scabrosus (Fr.) Karst, which were collected in Ailao Mountain, Yunnan, China in 2002 and identified by Ms. WANG Xianghua, Kunming Institute of Botany, Chinese Academy of Sciences. The compounds 2-7 are obtained by reacting sarcodonin A with dried Ac₂O or corresponding benzoyl chloride derivatives in the presence of 4-dimethylaminopyridine (DMAP) at room temperature. Specifically including:

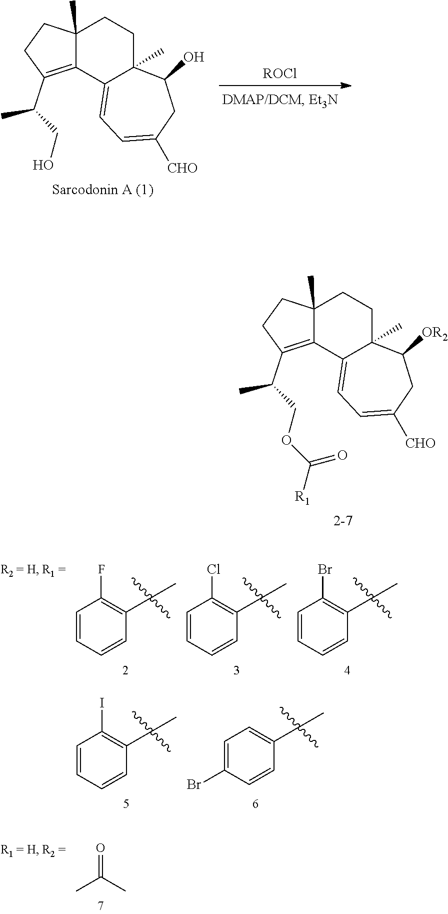

Scheme 1. Semisynthesis of sarcodonin A derivatives 2-7.

Preparation of the compounds according to the present invention, and determination and application of anti-neuroinflammation;

Experimental Materials

Experimental Cell Line:

BV-2 mouse microglia were purchased from the cell bank of Peking Union Medical College (Beijing, China).

Reagents and Instruments:

Commonly used biochemical reagents: high-sugar culture medium DMEM (Gibco, USA); fetal bovine serum (FBS) (Gibco, USA); lipopolysaccharide (LPS) (Sigma, USA); antibiotics (100 U/mL of streptomyces and penicillin) (HyClone, USA); cell culture bottles and petri dishes (Corning, China); pancreatic enzyme (Gibco, USA); sulfonyl rhodamine B (SRB); nitric oxide detection kit (Biyuntian, China); ELISA kit (TNFα, IL-1β, IL-6, IL-10) (R&D, USA); reverse transcription kit (Clontech, USA); real-time PCR kit (Roche, USA); primary antibody: anti-p-Tubulin, anti-GAPDH, anti-iNOS, anti-COX-2, anti-ARG-1, anti-ERK1/2, anti-phosphorylated ERK1/2, anti-p38 MAPK, anti-phosphorylated p38 MAPK, anti-JNK, anti-phosphorylated INK, anti-NF-κBp65 and anti-Lamin B1 (Abcam; Santa Cruz; CST, USA), peroxidase (HRP)-bound secondary antibody (Biyuntian, China), high-sensitivity ECL luminescent liquid (Biyuntian, China).

Commonly used instruments: superclean bench: SW-CJ-1F (Suzhou Antai Air Technology Co., Ltd.); vertical steam sterilizer: GI100T (Xiamen Zhiwei Instrument Co., Ltd.); carbon dioxide thermostatic cell incubator: Thermo forma 310 (Thermofisher Scientific, USA); water purifier: EQ 7000 (Merck, Germany); enzyme reader: Synergy HTX (BioTek Instrument Company, USA); ultraviolet-visible spectrophotometer: Nanodrop 2000 (ThermoFisher Scientific, USA); Real-time quantitative PCR instrument: Applied Biosystems QuantStudio 5 (Thermo Fisher Scientific, USA); PCR amplifier: C1000 (Bio-rad, USA); Chemiluminescence instrument: ChemiDocXRS+ (Bio-rad, USA).

1. Extraction and Separation of Sarcodonin A and Synthesis of Derivatives:

The dried fruiting bodies (10 kg) were ground and extracted with methanol for three times (10 L×3), and extracting solutions were concentrated under reduced pressure and combined (300 g), suspended with water, and extracted with ethyl acetate to obtain 200 g of paste and water-soluble part. The ethyl acetate part was mixed with crude silica gel (100-200 meshes), loaded on a silica gel column, subjected to gradient elution with a chloroform-acetone (9:1-1:9) mixed solvent, detected and combined by TLC, and divided into 9 components. Component 4 was subjected to silica gel column chromatography (CHCl₃-MeOH 98:2, 95:5, 90:10) to obtain five components (4.1-4.5). The component 4.1 was purified by a methanol gel column to obtain compound sarcodonin A(1) (250 mg).

15.6 mg (0.05 mmol) of sarcodonin A was dissolved in 3 mL of well-dried dichloromethane solvent, added with 20 μL of triethylamine and 2 mg of DMAP, stirred for 30 min, and slowly dropwise added with 1 mL (containing 8.8 mg, 0.05 mmol) of dichloromethane solution of o-fluorobenzoyl chloride for reaction at room temperature for 12 h, a raw material point detected by silica gel thin layer chromatography disappeared, 10 mL of water was added to quench the reaction, concentration under reduced pressure was performed to remove dichloromethane, extraction was performed with ethyl acetate (20 mL×3), an organic phase was washed with saturated ammonium chloride, water and saturated saline in turn, drying was performed with anhydrous magnesium sulfate, concentration under reduced pressure was performed to remove the solvent, and 45 mg of yellow-green resinous compound was obtained and isolated by silica gel (C-200-300, 6 mg) column chromatography (V petroleum ether:V ethyl acetate=10:1) to obtain 30 mg of yellow-green resinous compound 2 and the yield was 70.7%. A synthesis method for the compounds (3-6) was the same as above; 0.2 mmol of acyl chloride was added in the synthesis of the compound 7.

1.1 The Physical and Chemical Properties of 7 Cyathane Diterpenoids are:

The physical and chemical properties of the cyathane diterpenoid 1 according to the invention are:

C₂₀H₃₀O₄, yellow-green resin (CHCl₃); ESI-MS, 339.4 [M+Na]⁺; IR νₘₐₓ: 3406.9 (OH), 2930, 1657 (C═O), 1567, 1168, 1039, 751, 733 cm⁻¹. ¹³C-NMR (125 MHz, CDCl₃) δ: 194.41, 154.23, 146.69, 144.56, 141.09, 138.12, 19.87, 74.10, 66.44, 49.64, 48.40, 38.33, 36.42, 35.27, 33.71, 29.46, 29.26, 26.54, 24.36, 15.94; ¹H-NMR (500 MHz, CDCl₃) δ: 9.42 (s, 1H), 6.82 (dd, J=8.2, 2.5 Hz, 1H), 6.17 (d, J=8.2 Hz, 1H), 3.72 (br.d, J=4.6 Hz, 1H), 3.57 (dd, J=7.5, 2.8 Hz, 2H), 3.14 (dd, J=18.3, 5.8 Hz, 1H), 2.99-2.89 (m, 1H), 2.59-2.48 (m, 2H), 2.47-2.31 (m, 2H), 1.79-1.65 (m, 7H), 1.34 (dt, J=14.0, 3.6 Hz, 1H), 1.02 (s, 4H), 0.95 (s, 3H), 0.94 (d, J=6.9 Hz, 4H).

The physical and chemical properties of the cyathane diterpenoid 2 according to the invention are:

C₂₇H₃₁FO₄, yellow resin. IR (KBr): νₘₐₓ=2962, 1714, 1670, 1913, 1572, 1456, 1296, 1259, 1165, 1123, 1081, 1033, 1018, 780, 756 cm⁻¹; ¹H-NMR (500 MHz, CDCl₃) δ: 9.43 (s, 1H, CHO), 7.94 (td, J=7.6, 1.7 Hz, 1H), 7.56-7.47 (m, 1H), 7.21 (t, J=7.6 Hz, 1H), 7.15 (dd, J=10.3, 8.8 Hz, 1H), 6.77 (dd, J=8.2, 2.4 Hz, 1H), 6.14 (d, J=8.2 Hz, 1H), 4.38 (dd, J=10.7, 8.6 Hz, 1H), 4.23 (dd, J=10.8, 6.7 Hz, 1H), 3.73 (d, J=5.0 Hz, 1H), 3.34-3.20 (m, 1H), 3.16 (dd, J=18.2, 5.9 Hz, 1H), 2.60-2.50 (m, 2H), 2.50-2.44 (m, 2H), 1.78 (m, 1H), 1.74-1.65 (m, 3H), 1.36 (dt, J=14.0, 3.5 Hz, 1H), 1.07 (d, J=6.9 Hz, 3H), 0.99 (s, 3H), 0.98 (s, 3H); ¹³C-NMR (125 MHz, CDCl₃) δ: 194.2 (C═O), 164.4 (C═O of 2-fluorobenzol), 160.9, 153.9, 145.8, 144.4, 140.4, 138.0, 134.7, 134.6, 132.1, 124.1, 124.0, 119.8, 117.2, 117.0, 74.1, 68.2, 49.4, 48.3, 38.4, 36.4, 33.5, 32.0, 29.5, 29.1, 26.5, 23.7, 16.2; HR-ESI-MS: m/z 461.2094 [M+Na]⁺;

The physical and chemical properties of the cyathane diterpenoid 3 according to the invention are:

C₂₇H₃₁ClO₄, yellow resin. IR(KBr): νₘₐₓ=2931, 1729, 1668, 1570, 1455, 1431, 1290, 1247, 1166, 116, 1043, 1028, 744 cm⁻¹; ¹H-NMR (500 MHz, CDCl₃) δ: 9.41 (s, 1H, CHO), 7.77 (dd, J=7.3, 2.2 Hz, 1H), 7.68 (dd, J=7.6, 1.5 Hz, 1H), 7.44-7.30 (m, 2H), 6.75 (dd, J=8.2, 2.4 Hz, 1H), 6.10 (d, J=8.2 Hz, 1H), 4.37 (dd, J=10.7, 8.3 Hz, 1H), 4.24 (dd, J=10.8, 6.9 Hz, 1H), 3.72 (d, J=5.1 Hz, 1H), 3.28-3.18 (m, 1H), 3.15 (dd, J=18.3, 5.8 Hz, 1H), 2.60-2.44 (m, 4H), 1.79 (ddd, J=12.6, 7.7, 4.9 Hz, 1H), 1.75-1.65 (m, 5H), 1.35 (dt, J=13.9, 3.6 Hz, 1H), 1.07 (d, J=6.9 Hz, 3H), 1.00 (s, 3H), 0.97 (s, 3H); ¹³C-NMR (125 MHz, CDCl₃) δ: 194.3 (C═O), 166.2 (C═O of 2-chlorobenzoyl), 154.0, 146.0, 144.4, 140.5, 138.2, 134.6, 132.8, 132.3, 131.3, 127.4, 121.8, 120.0, 74.1, 68.6, 49.5, 48.4, 38.5, 36.5, 33.7, 32.2, 29.7, 29.2, 26.6, 24.0, 16.5; HR-ESI-MS: m/z 477.1801 [M+Na]⁺;

The physical and chemical properties of the cyathane diterpenoid 4 according to the invention are:

C₂₇H₃₁BrO₄, yellow resin. IR(KBr): νₘₐₓ=2931, 1778, 1668, 1571, 1435, 1369, 1292, 1247, 1166, 1116, 1049, 1014, 747, 719 cm⁻¹; ¹H-NMR (500 MHz, CDCl₃) δ: 9.41 (s, 1H, CHO), 7.81 (dd, J=7.8, 1.5 Hz, 1H), 7.46 (td, J=7.8, 1.4 Hz, 1H), 7.42 (dd, J=8.1, 1.6 Hz, 1H), 7.32 (td, J=7.7, 1.4 Hz, 1H), 6.74 (dd, J=8.2, 2.5 Hz, 1H), 6.10 (d, J=8.2 Hz, 1H), 4.37 (dd, J=10.7, 8.4 Hz, 1H), 4.24 (dd, J=10.8, 6.9 Hz, 1H), 3.72 (d, J=5.0 Hz, 1H), 3.30-3.19 (m, 1H), 3.15 (dd, J=18.3, 5.8 Hz, 1H), 2.57-2.50 (m, 2H), 2.49-2.44 (m, 2H), 1.79 (ddd, J=12.7, 7.7, 4.9 Hz, 1H), 1.75-1.67 (m, 3H), 1.35 (dt, J=13.9, 3.7 Hz, 1H), 1.07 (d, J=6.9 Hz, 3H), 1.00 (s, 3H), 0.97 (s, 3H); ¹³C-NMR (126 MHz, CDCl₃) δ: 194.3 (C═O), 165.8 (C═O of 2-bromobenzoyl), 154.0, 145.9, 144.4, 140.5, 138.2, 133.8, 132.8, 131.5, 131.4, 130.2, 126.8, 120.0, 74.1, 68.6, 49.6, 48.4, 38.5, 36.6, 33.7, 32.2, 29.6, 29.2, 26.6, 24.0, 16.4; HR-ESI-MS: m/z 521.1290[M+Na]⁺;

The physical and chemical properties of the cyathane diterpenoid 5 according to the invention are:

C₂₇H₃₁IO₄, yellow resin. IR(KBr): νₘₐₓ=2933, 1725, 1666, 1570, 1428, 1246, 1116, 1133, 1099, 1043, 1015, 741 cm⁻¹. ¹H-NMR (500 MHz, CDCl₃) δ: 9.42 (s, 1H, CHO), 8.01 (dd, J=7.9, 0.6 Hz, 1H), 7.79 (dd, J=7.8, 1.6 Hz, 1H), 7.49-7.33 (m, 1H), 7.16 (td, J=7.7, 1.6 Hz, 1H), 6.77 (dd, J=8.2, 2.4 Hz, 1H), 6.09 (d, J=8.2 Hz, 1H), 4.36 (dd, J=10.7, 8.2 Hz, 1H), 4.24 (dd, J=10.7, 7.0 Hz, 1H), 3.72 (d, J=5.0 Hz, 1H), 3.28-3.19 (m, 1H), 3.15 (dd, J=18.3, 5.8 Hz, 1H), 2.63-2.44 (m, 1H), 1.79 (ddd, J=12.6, 7.8, 4.7 Hz, 1H), 1.75-1.64 (m, 1H), 1.35 (dt, J=14.0, 3.7 Hz, 1H), 1.07 (d, J=6.8 Hz, 1H), 1.01 (s, 1H), 0.97 (s, 1H); ¹³C-NMR (126 MHz, CDCl₃) δ: 194.3 (C═O), 166.4 (C═O of 2-iodobenzoyl), 154.0, 146.0, 144.4, 141.7, 140.4, 138.2, 135.1, 132.9, 130.9, 128.1, 120.0, 94.3, 74.1, 68.6, 49.5, 48.4, 38.5, 36.5, 33.7, 32.2, 29.7, 29.2, 26.6, 24.0, 16.46; HR-ESI-MS:m/z 569.1147 [M+Na]⁺;

The physical and chemical properties of the cyathane diterpenoid 6 according to the invention are:

C₂₇H₃₁BrO₄, yellow resin. IR(KBr): νₘₐₓ=2937, 1737, 1672, 1577, 1371, 1229, 1166, 1022 cm-1; ¹H-NMR (500 MHz, CDCl₃) δ: 9.43 (s, 1H, CHO), 7.88 (d, J=8.5 Hz, 1H), 7.59 (d, J=8.5 Hz, 1H), 6.73 (dd, J=8.2, 2.4 Hz, 1H), 6.08 (d, J=8.2 Hz, 1H), 4.28 (dd, J=16.1, 7.6 Hz, 1H), 3.74 (d, J=5.3 Hz, 1H), 3.29-3.19 (m, 1H), 3.18 (d, J=5.8 Hz, 1H), 2.59-2.43 (m, 1H), 2.17 (s, 1H), 1.84-1.72 (m, 1H), 1.72-1.58 (m, 2H), 1.36 (dt, J=13.9, 3.6 Hz, 1H), 1.06 (d, J=6.9 Hz, 1H), 0.99 (s, 1H), 0.98 (s, 1H); ¹³C-NMR (126 MHz, CDCl₃) δ: 194.2, 165.8, 154.2, 146.1, 144.2, 140.4, 138.3, 132.0, 131.1, 129.3, 128.4, 119.8, 74.2, 68.2, 49.6, 48.5, 38.5, 36.5, 33.6, 32.2, 29.7, 29.2, 26.6, 24.0, 16.3; HR-ESI-MS:m/z 521.1290 [M+Na]⁺;

The physical and chemical properties of the cyathane diterpenoid 7 according to the invention are:

C₂₄H₃₂O₅, yellow resin. IR(KBr): νₘₐₓ=2937, 1737, 1672, 1577, 1371, 1229, 1166, 1022 cm⁻¹; ¹H-NMR (500 MHz, CDCl₃) δ:9.40 (s, 1H, CHO), 6.75 (dd, J=8.1, 2.2 Hz, 1H), 6.05 (d, J=8.1 Hz, 1H), 4.97 (d, J=5.6 Hz, 1H), 4.14 (dd, J=10.7, 7.9 Hz, 1H), 3.91 (dd, J=10.7, 7.2 Hz, 1H), 3.69 (d, J=5.3 Hz, 1H), 3.18 (dd, J=18.1, 6.3 Hz, 1H), 3.12-3.00 (m, 1H), 2.49 (d, J=18.2 Hz, 1H), 2.45-2.32 (m, 3H), 2.06 (s, 3H), 2.06-1.98 (m, 3H), 1.96 (s, 4H), 1.76 (ddd, J=12.3, 8.5, 3.7 Hz, 2H), 1.71-1.64 (m, 3H), 1.60 (ddd, J=13.2, 4.7, 3.2 Hz, 2H), 1.39-1.34 (m, 1H), 1.02 (s, 4H), 0.99 (d, J=6.9 Hz, 4H), 0.97 (s, 4H); ¹³C-NMR (126 MHz, CDCl₃) δ:193.8, 171.0, 170.4, 152.6, 145.6, 144.9, 139.8, 138.3, 120.5, 75.4, 67.5, 49.5, 46.6, 38.6, 36.2, 33.3, 32.1, 29.5, 26.2, 23.8, 21.1, 21.1, 16.2; ESI-MS (positive) m/z: 423.2117 [M+Na]⁺.

2 Cell Culture

The mouse microglia (BV-2) were cultured in the DMEM culture medium containing 10% of fetal bovine serum (FBS) and 1% of antibiotics (100 U/mL of streptomyces and penicillin) at 37° C. in the incubator of 5% CO₂.

3 Determination of Anti-Neuroinflammatory Activity of LPS-Induced BV-2 Cells

3.1 Cell Vitality Test

The viability of the BV-2 cells was measured by the SRB method. The cells with a density of 1.5×10⁴ cells/mL were incubated in a 96-well plate until they attached to the wall for 24 h. The culture medium was replaced with a fresh culture medium containing indicated compounds, and cultured for another 24 h. Then the cells were fixed with trichloroacetic acid (10%, w/v) for 5 minutes, and the excess acid was washed with distilled water for 5 times. The cells were treated with SRB (0.4%, w/v) for 30 minutes, and excess dye was washed with trichloroacetic acid (1%, w/v). The bound dye was dissolved in TBS (10 mM, pH 10.5), and the absorbance was measured at 510 nm on a Bio-Tek micropore plate reader. The absorbance of the DMSO control was assumed as 100%.

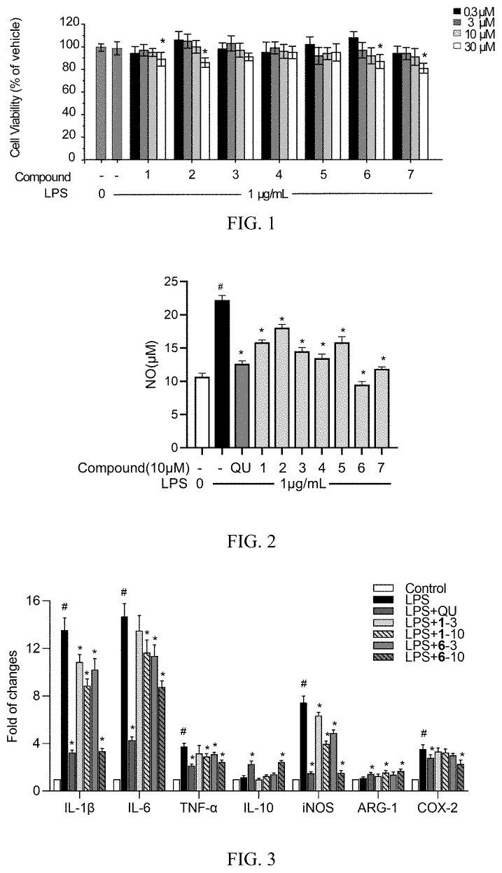

As shown in FIG. 1, the compounds below 10 μM do not affect survival of the BV-2 cells, so that the maximum concentration of 10 μM is selected for further studies.

3.2 Inhibition of Nitric Oxide (NO) Production

NO is an important inflammatory mediator. Excessive production of NO is common in neurons and glial cells of brains with ND^{[8]}. Griess reagent was configured to evaluate the influence on NO production in order to measure the anti-inflammatory effects of sarcodonin A and its derivative in LPS-induced BV-2 microglia. Before adding the detection reagent, BV-2 cells (2×10⁵/mL) were incubated in the 96-well plate and cultured at 37° C. for 24 h. The experimental groups were divided into a control group (DMSO), an LPS-treated group (1 g/mL of LPS) and a compound-treated group (1 μg/mL of LPS+10 μM compound), and then incubation was performed for 24 h. The nitrite concentration in the culture medium was measured by a commercial analysis kit. The main principle was based on the griess method. Supernatant of the BV-2 cells (50 μL) reacted with the griess reagent (50 μL of griess regent I and 50 μL of griess regent II). The absorbance was measured at 540 nm on the Bio-Tek micropore plate reader, and the nitrite concentration was calculated by a nitrite standard curve with sodium nitrite as the standard.

The inhibition rate %=(A_{LPS}−A_{compound})/(A_{LPS}−A_{blank})×100;

As shown in FIG. 2, after the LPS stimulation for 24 h, the production of NO in BV-2 cells significantly increases by about 2.5 times. Sarcodonin A and its derivatives can inhibit the production of NO in different degrees. In all halogenated 19-O-benzoyl derivatives, 3, 4 and 6 have higher inhibitory activity on NO production, even better than the parent compound sarcodonin A. Compared with the LPS control group, the inhibitory activity of brominated 19-O-benzoyl derivative (6) is the highest and is 55%, consistent with the fact that o-bromo-substituted 19-O-benzoyl derivative (4) has the highest inhibitory activity in o-halogen-substituted 19-O-benzoyl derivatives (2-5). It is worth noting that the 14,19-O-acetyl derivative (7) also showed a strong inhibitory effect similar to that of 6.

Therefore, the preliminary SAR study shows that 19-hydroxyl and 14-hydroxyl can be modified to enhance the anti-inflammatory activity of sarcodonin A.
[8] F. J. Jimenez-Jimenez, H. Alonso-Navarro, M. T. Herrero, E. Garcia-Martin, J. A. Agundez, 2016. An Update on the Role of Nitric Oxide in the Neurodegenerative Processes of Parkinson's Disease, Curr. Med. Chem. 23(24):2666-2679.

4 Effects of Compounds 1 and 6 on mRNA Levels of M1/M2 Biomarkers in LPS-Induced BV-2 Cells

In order to detect the regulation of M1/M2 phenotype switch by 1 and 6, the effects on the mRNA expression of representative M1 and M2 biomarkers in the LPS-stimulated BV-2 cells were evaluated. The cells were incubated into a 6 cm of culture dish (5 mL/dish) at a density of 2×10⁵ cfu, and cultured in a constant-temperature incubator of 5% CO₂ at 37° C. for 12 h. According to the manufacturer's instructions, total RNA was extracted by Trizol, and concentration and purity thereof were measured by a UV-Vis spectrophotometer. 2 μg of isolated RNA was reversely transcribed into cDNA by Prime Script RTMaster Mix kit. By using a Syr Green quantitative RT-PCR kit, an mRNA level was evaluated by qRT-PCR response. β-actin was used as an internal standard. The primer sequences are as follows (Table 1).

TABLE 1
Primer sequences of real-time quantitative PCR

| | | Primer (5′-3′) |
|---|---|---|
| Gene | Accession ID | F, forward; R, reverse |
| IL-1β | XM_006498795.3 | F: CGCAGCAGCACATCAACAAGAGC |
| | | R: TGTCCTCATCCTGGAAGGTCCACG |
| IL-6 | NM_010548.2 | F: CCAGAGATACAAAGAAATGATGG |
| | | R: ACTCCAGAAGACCAGAGGAAA |
| TNF-α | NM_001278601.1 | F: AGCCCCCAGTCTGTATCCTT |
| | | R: ACAGTCCAGGTCACTGTCCC |
| IL-10 | NM_010548.2 | F: GCTCTTACTGACTGGCATGAG |
| | | R: CGCAGCTCTAGGAGCATGTG |
| iNOS | XM_006532446.3 | F: TGGAGCGAGTTGTGGATTGTC |
| | | R: GGTCGTAATGTCCAGGAAGTAG |
| COX-2 | NM_011198.4 | F: CCAGCACTTCACCCATCAGT |
| | | R: ACACCTCTCCACCAATGACC |
| ARG-1 | NM_007482.3 | F: CGCCTTTCTCAAAAGGACAG |
| | | R: CCAGCTCTTCATTGGCTTTC |
| β-actin | NM_007393 | F: GGCATCGTGATGGACTCCG |
| | | R: GCTGGAAGGTGGACAGCGA |

As shown in FIG. 3, the LPS significantly increases the mRNA level of the M1 markers (including IL-1β, IL-6, TNF-α, iNOS and COX-2), but hardly affects the mRNA expression of the M2 markers (including IL-10 and ARG-1). However, not only 6 inhibits the mRNA expression of IL-1β, iNOS and TNF-α induced by the LPS, but also IL-6 inhibits the mRNA expression of COX-2 induced by the LPS at 10 μM, and also promotes the mRNA levels of IL-10 and ARG-1 when the concentration is increased to 10 μm. IL-6 inhibits the mRNA expression of IL-1β and iNOS induced by the LPS in a dose-dependent manner. At the same time, 1 inhibits the mRNA expression of TNF-α and IL-6 induced by the LPS at 10 μM, and enhance the mRNA level of ARG-1. For IL-10 and COX-2, 1 does not affect the expression of these genes at the tested concentration. The results show that 6 may regulate the M1/M2 phenotype switch of the microglia more effectively, which is consistent with Griess analysis. This suggests that 1 and 6 may reverse M1 polarization of the LPS-induced microglia, thereby showing the anti-inflammatory effect.

5 Effects of Compounds 1 and 6 on M1/M2 Marker Cytokines Secretion in LPS-Induced BV-2 Cells

To further evaluate the role of 1 and 6 in M1 polarization of the LPS-induced microglia, the release of pro-inflammatory M1 cytokines (including TNF-α, IL-6 and IL-1β) and anti-inflammatory M2 cytokines (including IL-10) in the LPS-induced BV-2 cells was detected by ELISA. The BV-2 cells were incubated in a 6-well plate (2 mL/well) at a density of 2×10⁵ cfu, cultured in a constant-temperature incubator of 5% CO₂ at 37° C. for 24 h, then pretreated with different concentrations of compounds 1 and 6 (3 μM, 10 μM), LPS (1 μg/mL) and DMSO for 1 h, and then induced by the LPS for 24 h. Supernatant of the cell culture medium was collected, and the concentrations of TNF-α, IL-6, IL-10 and IL-1β were measured according to manufacturer's instructions.

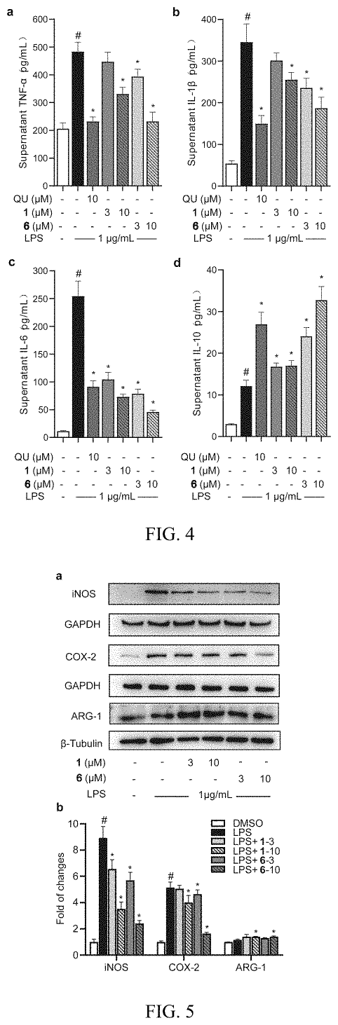

As shown in FIG. 4, the LPS significantly up-regulates IL-6, IL-1β and TNF-α, but slightly enhances the production of IL-10. As the concentration increases, the inhibitory effect of 1 and 6 on the production of IL-6, IL-1β and TNF-α and the promoting effect on the production of IL-10 was enhanced. At the same concentration, 6 is more effective than 1. The research results are consistent with the analysis results of griess and qRT-PCR, which confirms that 6 is more active than the parent compound 1 in reversing the M1 polarization of the LPS-induced BV-2 microglia.

6 Effects of Compounds 1 and 6 on M1/M2 Marker Protein Expression in LPS-Induced BV-2 Cells

The protein expression of iNOS, COX-2(M1 marker) and ARG-1(M2 marker) was analyzed by Western blotting. The BV-2 cells (5×10⁵ cfu) cultured in a 6 cm dish were pretreated with 1 or 6 (3.10 μM) respectively for 1 h, and then 1 μg/ml LPS was added for 24 h. The cells were lysed in an ice-cold RIPA lysis buffer with a mixed solution of 10 mM of PMSF and 1% of protein inhibitors. The cytosol lysate was centrifuged at 4° C. at 12000 rpm for 30 minutes. Total protein was isolated from the lysate and their concentration was determined by BCA.

A sample was separated by 8%-12% SDS-PAGE and transferred to a 0.45 m PVDF membrane. The membrane was blocked with 5% BSA in TBST (20 mM of Tris-HCl, 150 mM of NaCl, 0.1% of Tween-20) for 1 h at room temperature, and then incubated with appropriate primary antibodies: anti-β-tubulin (1:1000), anti-GAPDH (1:1000), anti-iNOS (1:500), anti-COX-2 (1:1000) and anti-ARG-1 (1:500) at 4° C. overnight. The membrane was washed in the TBST for 3×5 minutes, and then incubated with the secondary antibody bound to appropriate peroxidase (HRP) and left at room temperature for 1 h. The blot was washed in the TBST for 3×5 minutes and exposed to an ECL chemiluminescence reagent. At last, the signal was detected by the Bio-Rad ChemiDoc XRS+ system.

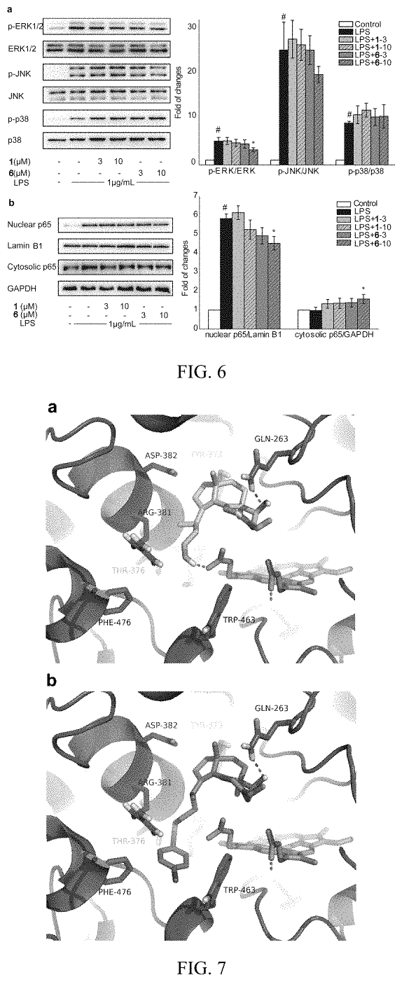

As shown in FIG. 5, the LPS significantly enhances the expression of iNOS and COX-2, but has little effect on the expression of ARG-1. 1 inhibits the expression of LPS-induced iNOS and promotes the expression of ARG-1 in a dose-dependent manner. As expected, 6 has more obvious inhibitory effect on the expression of iNOS and COX-2, and more obvious promoting effect on the expression of ARG-1. In a word, 1 and 6 inhibit the M1 polarization and enhance M2 polarization of the LPS-induced BV-2 cells.

7 Compound 6 Inhibits the M1 Polarization of the Microglia by Inhibiting MAPK Activation in the LPS-Induced BV-2 Cells.

Previous studies have shown that the over-activation of MAPK signaling cascade is related to neuroinflammatory reaction^{[9-10]}. In order to clarify the action mode of 6 in reversing the M1 polarization of the microglia, the effects of 6 on phosphorylation levels of ERK1/2, INK and p38 MAPK in the LPS-induced BV-2 microglia are further studied. BV-2 cells (5×10⁵ cfu) cultured in a 6 cm dish are pretreated with compound 1 or 6 (3.10 μM) respectively for 1 h, and then added with the LPS (1 μg/mL) for 30 min. The similar method is used by Western blotting, and the primary antibodies are: anti-p-tubulin (1:1000), anti-GAPDH (1:1000), anti-ERK1/2 (1:1000), anti-phosphorylated ERK1/2 (1:1000), anti-p38 MAPK (1:2000), anti-phosphorylated p38 MAPK (1:1000), anti-JNK (1:2000) and anti-phosphorylated JNK (1:1000).

As shown in FIG. 6, the LPS significantly upregulates the phosphorylation levels of ERK1/2, INK and p38 MAPK. 6 significantly inhibits the activation of ERK1/2 and INK induced by the LPS, but has little effect on the phosphorylation of p38 MAPK. The results show that 6 can antagonize the activation of MAP kinase induced by the LPS, especially ERK1/2 and JNK. MAPK plays an important role in the synthesis of inflammatory cytokines.

The activation of MAPK directly affects NF-κB-mediated transcription of inflammatory factors. Therefore, 6 antagonizes the activation of ERK1/2 and JNK induced by the LPS, which possibly leads to a decrease in the production of pro-inflammatory molecules, thereby hindering the dominant M1 phenotypic polarization.
[12] B. Dinda, M. Dinda, G. Kulsi, A. Chakraborty, S. Dinda, 2019. Therapeutic potentials of plant iridoids in Alzheimer's and Parkinson's diseases: A review, Eur J Med Chem 169, 185-199.
[10] R. Dhapola, S. S. Hota, P. Sarma, A. Bhattacharyya, B. Medhi, D. H. Reddy, 2021. Recent advances in molecular pathways and therapeutic implications targeting neuroinflammation for Alzheimer's disease, Inflammopharmacology 29(6).

8 Compound 6 Blocks Nuclear Translocation of NF-1B in the LPS-Induced BV-2 Cells to Reverse the M1 Polarization of Microglia.

NF-κB is a transcription factor with good characteristics, and is considered to be the key factor of microglia-mediated neuroinflammation^{[11]}. Normally, NF-κB, as p50/p65/IκB trimer, is bound to inhibitor protein IκB and confined in the cytoplasm. Once activated by various stimuli such as the LPS, the molecule is subjected to nuclear shift. In the nucleus, NF-κB initiates the gene transcription of related inflammatory factors^{[12]}.

The nuclear translocation of NF-κB subunit p65 as an activation index of NF-κB is examined. The cells were collected in the same way for Western blotting. A extraction kit is configured to isolate and extract nuclear and cytoplasmic parts according to the instructions. The primary antibodies are anti-NF-κB p65 (1:1000), anti-Lamin B1 (1:1000) and anti-GAPDH (1:1000).

As shown in FIG. 7, the LPS significantly increases NF-κB p65 in the nucleus, but decreases NF-κB p65 in the cytoplasm component. Treatment of 6 significantly inhibits the ability of the LPS to stimulate nuclear translocation of NF-κB p65. In fact, it is reported that NF-κB is the key signal to regulate the M1/M2 balance of the microglia. The loss of this function leads to the selective susceptibility of CNS to chronic neuroinflammation^{[13]}.
[14] S. S. Singh, S. N. Rai, H. Birla, W. Zahra, A. S. Rathore, S. P. Singh, 2020. NF-κB-Mediated Neuroinflammation in Parkinson's Disease and Potential Therapeutic Effect of Polyphenols, Neurotox Res 37(3):491-507.
[15] C. Ju Hwang, D. Y. Choi, M. H. Park, J. T. Hong, 2019. NF-κB as a Key Mediator of Brain Inflammation in Alzheimer's Disease, CNS Neurol Disord Drug Targets 18(1):3-10.
[16] T. Taetzsch, S. Levesque, C. McGraw, S. Brookins, R. Luqa, M. G. Bonini, R. P. Mason, U. Oh, M. L. Block, 2015. Redox regulation of NF-κB p50 and M1 polarization in microglia, Glia 63(3):423-40.

9 Binding of 1 and 6 to iNOS by Molecular Simulation

L-arginine mainly produces NO in the immune response to the stimuli such as the LPS through inducible nitric oxide synthase^{[14]}. In order to understand the inhibition of sarcodonin A compound on the LPS-induced NO production molecularly, molecular docking is conducted to study the interaction between 1 and 6 and iNOS. As shown in FIG. 8, both 1 and 6 are tightly bound to an active cavity of iNOS and form a hydrogen bond interaction. In addition, the 19 benzoyl ring of 6 has hydrophobic interaction with Phe476 and Trp463 in different iNOS chains, thus forming stronger interaction with iNOS, which is consistent with free binding energy of 1 and 6. Therefore, sarcodonin A compound may inhibit the production of NO by occupying active sites of iNOS, which is consistent with previous research on other anti-neuroinflammatory cyathane diterpenoids from mushroom Cyathus africanus^{[15,16]}.
[17] H. Possel, H. Noack, J. Putzke, G. Wolf, H. Sies, 2000. Selective upregulation of inducible nitric oxide synthase (iNOS) by lipopolysaccharide (LPS) and cytokines in microglia: in vitro and in vivo studies, Glia 32(1):51-9.
[18] J. Wei, Y. Cheng, W. H. Guo, D. C. Wang, Q. Zhang, D. Li, J. Rong, J. M. Gao, 2017. Molecular Diversity and Potential Anti-neuroinflammatory Activities of Cyathane Diterpenoids from the Basidiomycete Cyathus africanus, Sci Rep 7(1)8883.
[19] J. Wei, W. H. Guo, C. Y. Cao, R. W. Kou, Y. Z. Xu, M. Górecki, L. Di Bari, G. Pescitelli, J. M. Gao, 2018. Polyoxygenated cyathane diterpenoids from the mushroom Cyathus africanus, and their neurotrophic and anti-neuroinflammatory activities, Sci Rep 8(1):2175.

In summary, in the aspect of prevention and treatment of the neurodegenerative diseases, the cyathane diterpenoids can be developed as potential lead compounds.

The preferred embodiments of the present disclosure have been described in detail in combination with the drawings above, but the present disclosure is not limited to the specific details of the above embodiments. Within a technical concept scope of the present disclosure, many simple transformations can be made to the technical solutions of the present disclosure, and these simple transformations all belong to the scope of protection of the present disclosure.

In addition, it should be noted that respective specific technical features described in the above specific embodiments can be combined in any suitable way without contradiction. In order to avoid unnecessary repetitions, the present disclosure will not separately explain various possible combinations.

In addition, any combination of different embodiments of the present disclosure can also be made, as long as the combination does not violate the idea of the present disclosure, it should also be regarded as the content of the present disclosure.

What is claimed is:

1. The cyathane diterpenoid sarcodonin A numbered as 1 is isolated from fruiting bodies of Sarcodon scabrosus (Fr.) Karst and the derivatives of sarcodonin A numbered as 2-7 are synthesized;

2. According to claim 1, the cyathane diterpenoids 1-7 exhibit anti-neuroinflammatory effects by reversing M1/M2 microglia polarization through MAPK/NF-κB signaling pathway and thus the cyathane diterpenoids, hydrates thereof, pharmaceutically acceptable salts thereof, pharmaceutically acceptable carriers thereof and/or compositions containing cyathane diterpenoids can be applicated in preparation of drugs for treating neuroinflammation-associated neurodegenerative diseases comprising but not limited to Alzheimer's disease, Amyotrophic lateral sclerosis, Huntington's disease or Parkinson's disease.
